(19) United States
(12) Patent Application Publication
(10) Pub. No.: US 2026/0216472 A1
(43) Pub. Date: Jul. 30, 2026

(54) METHOD FOR PRODUCING AN ABLATION CATHETER COMPRISING FLUSHING PASSAGES

(71) Applicants: Heraeus Medevio GmbH & Co. KG, Hanau (DE); Contract Medical International GmbH, Dresden (DE)

(72) Inventors: Ilias NIKOLAIDIS, Hanau (DE); Mario HEINTZE, Dresden (DE); Llewellyn Wallace GROENEVELD, Dresden (DE); Raffael KUNZ, Dresden (DE)

(21) Appl. No.: 19/452,694

(22) Filed: Jan. 19, 2026

(30) Foreign Application Priority Data
Jan. 24, 2025 (DE) 102025102559.2

(51) Int. Cl. A61M 25/00; A61B 18/14; A61B 18/00

(52) U.S. Cl. CPC A61M 25/0015; A61B 18/1492; A61B 2018/00077; A61B 2018/00083; A61B 2018/00178; A61B 2018/00577; A61B 2218/002

(57) ABSTRACT

A method producing a catheter body comprising flushing passages, including steps of: a) providing a hollow-cylindrical inner liner with an inner lumen and a radially outer inner liner outer surface; b) providing a flexible circuit board comprising a plurality of electrical conductors; c) arranging the flexible circuit board on the inner liner outer surface; d) sheathing the inner liner and the flexible circuit board with an outer insulation; e) establishing a connection between the inner liner and the outer insulation so that the flexible circuit board is fixed between the inner liner and the outer insulation; f) providing at least one electrode; g) establishing electrical contact between the at least one electrode and at least one of the electrical conductors of the flexible circuit board; and, h) creating a plurality of flushing passages which fluidically connect the inner lumen to a surface of the catheter body.

### CROSS-REFERENCE TO RELATED APPLICATION

This application claims priority pursuant to 35 U.S.C. 119(a) to German Patent Application No. 102025102559.2, filed January 24, 2025, which application is incorporated herein by reference in its entirety.

### FIELD OF THE INVENTION

The invention relates to a method for producing a catheter body, in particular for an ablation catheter, that comprises flushing passages, comprising the method steps of:

a) providing a hollow-cylindrical inner liner with an inner lumen and a radially outer inner liner outer surface;

b) providing a flexible circuit board comprising a plurality of electrical conductors;

c) arranging the flexible circuit board on the inner liner outer surface;

d) sheathing the inner liner and the flexible circuit board with an outer insulation;

e) establishing a connection between the inner liner and the outer insulation so that the flexible circuit board is fixed between the inner liner and the outer insulation;

f) providing at least one electrode;

g) establishing electrical contact between the at least one electrode and at least one of the electrical conductors of the flexible circuit board; and,

h) creating a plurality of flushing passages which fluidically connect the inner lumen to a surface of the catheter body.

The invention further relates to a catheter body produced by such a method.

### BACKGROUND

Catheters have been used in medical procedures for many years. Among other things, catheters can be used in medical procedures to examine, diagnose, and/or treat tissue in areas of the body such as blood vessels that are not easily accessible without more invasive procedures. For example, catheters can be used to deliver electrical energy to selected areas of the human body, such as the heart, to kill specific tissue, such as cardiac tissue. This is often referred to as “ablation.” Catheters can also be used to stimulate body tissue. During stimulation, the amount of energy transferred to the body tissue is usually lower than during ablation.

Another procedure, often called "mapping," uses a catheter comprising sensing electrodes to monitor various forms of bioelectrical activity in the human body.

Catheters can therefore be used both to transmit (during “ablation” and “stimulation”) and to absorb (during “mapping”) energy to and from body tissue.

Regardless of the direction of energy flow, catheters often contain a plurality of electrodes, i.e., two or more, at a distal catheter body end region, wherein the distal catheter body end region often describes the end that is inserted into the patient. Typically, the catheter contains the same number of electrodes at its proximal catheter body end region, which are electrically connected to the electrodes at the distal catheter body end region. The electrodes at the proximal catheter body end region are used to connect electrical devices according to the desired areas of application of the catheter.

Particularly in the area of ablation catheters, such as PFA catheters (pulmonary vein ablation catheters), which are generally used in certain cardiac treatments, the catheter bodies often comprise flushing passages for draining a fluid, such as a cooling liquid, from the catheter. These flushing passages are used in particular to cool the catheter and the surrounding tissue in order to reduce unwanted tissue damage caused by overheating during ablation. In addition, draining a fluid can prevent the formation of blood clots, which can be further improved by the simultaneous administration of anticoagulants via the fluid.

The production of such a catheter comprising flushing passages is usually complex and costly. The electrodes required for ablation, in particular ring electrodes, are usually electrically conductively connected with wires or cables that run in an inner lumen of the catheter body, depending upon their intended use. However, these wires or cables running in the inner lumen have some disadvantages. Firstly, they take up a significant part of the diameter of the inner lumen, which, depending upon the intended further use of the inner lumen, e.g., for a guide wire, requires a larger overall diameter of the catheter body. Larger overall diameters are disadvantageous because they could damage the patient's blood vessels during insertion.

In addition, the wires or cables running in the inner lumen make it difficult to form, especially subsequently form, the flushing passages in the pre-assembled catheter body, since their exact position and orientation in the inner lumen is not known and can also change - for example, if the catheter body is bent. This means that the formation of the flushing passages, e.g., by forming a hole through the wall of the catheter body, always carries the risk of damaging one of the cables or wires when the electrodes are already electrically contacted.

Therefore, the flushing passages are usually already formed in electrodes, some of which are designed for this purpose, before the electrodes are mounted on the catheter body, which requires complex manufacturing. In addition, these special electrodes still must be aligned with respect to prefabricated flushing passages in the wall of the catheter body, which further complicates mounting thereof. This manufacturing method is therefore expensive, technically demanding, and inflexible regarding the number and location of the flushing passages. Such specially shaped electrodes are described, for example, in US Patent No. 10,856,575 B2.

There is therefore a need in the market for an improved manufacturing method for catheter bodies, in particular for catheter bodies for ablation catheters, comprising flushing passages.

### OBJECTS

One object of the present invention is to overcome, at least in part, one or more of the disadvantages resulting from the prior art.

In particular, one object of the present invention is to provide a method for producing a catheter body, in particular a catheter body for an ablation catheter, comprising flushing passages, which method allows for the simple, safe, cost-effective, and flexible production of such a catheter body. The method is intended to be able to form a plurality of, i.e., at least two, flushing passages in the catheter body in a flexible and safe-to-use manner, especially after electrical contact has been established with the catheter electrodes.

Furthermore, the method is intended to allow for the production of a catheter body with the smallest possible overall diameter.

Another object of the present invention is to provide a catheter body which can be used cost-effectively and as flexibly as possible. Furthermore, the catheter body is to be as cost-effective to produce as possible.

### PREFERRED EMBODIMENTS OF THE INVENTION

A contribution to the at least partial fulfillment of at least one of the aforementioned objects is made by the features of the independent claims. The dependent claims provide preferred embodiments that contribute to the at least partial fulfillment of at least one of the objects.

A first embodiment of the invention is a method for producing a catheter body, in particular for an ablation catheter, that comprises flushing passages, comprising the method steps of:

a) providing a hollow-cylindrical inner liner with an inner lumen and a radially outer inner liner outer surface;

b) providing a flexible circuit board comprising a plurality of electrical conductors;

c) arranging the flexible circuit board on the inner liner outer surface;

d) sheathing the inner liner and the flexible circuit board with an outer insulation;

e) establishing a connection between the inner liner and the outer insulation so that the flexible circuit board is fixed between the inner liner and the outer insulation;

f) providing at least one electrode;

g) establishing electrical contact between the at least one electrode and at least one of the electrical conductors of the flexible circuit board; and,

h) creating a plurality of flushing passages which fluidically connect the inner lumen to a surface of the catheter body.

In a preferred embodiment of the method, method step e) is carried out before method steps f) and g). This embodiment is a second embodiment of the invention, which is preferably dependent upon the first embodiment of the invention.

In a preferred embodiment of the method, method step e) is carried out before method step h). This embodiment is a third embodiment of the invention, which is preferably dependent upon the first or second embodiment of the invention.

In a preferred embodiment of the method, method step e) is carried out before method steps f) and g), and method step h) is carried out after method steps f) and g). This embodiment is a fourth embodiment of the invention, which is preferably dependent upon one of the preceding embodiments of the invention.

In a preferred embodiment of the method, at least one contact opening is created in the outer insulation for the establishment of electrical contact with the electrode in method step g) so that electrical contact is selectively established between the electrode and at least one electrical conductor of the flexible circuit board that is accessible via the contact opening. This embodiment is a fifth embodiment of the invention, which is preferably dependent upon one of the preceding embodiments of the invention.

In a preferred embodiment of the method, for the establishment of the connection between the inner liner and the outer insulation in method step e), the inner liner and/or the outer insulation is/are warmed up, in other words heated, forming an integral bond between the inner liner, in particular the inner liner outer surface, and the outer insulation. This embodiment is a sixth embodiment of the invention, which is preferably dependent upon one of the preceding embodiments of the invention.

In a preferred embodiment of the method, a laser, or in other words a laser device, is used to generate the plurality of flushing passages in method step h). This embodiment is a seventh embodiment of the invention, which is preferably dependent upon one of the preceding embodiments of the invention.

In a preferred embodiment of the method, flushing passages are produced in method step h), which extend at least through the at least one electrode and the inner liner. This embodiment is an eighth embodiment of the invention, which is preferably dependent upon one of the preceding embodiments of the invention.

In a preferred embodiment of the method, flushing passages are produced in method claim h), which extend at least through the outer insulation and the inner liner. This embodiment is a ninth embodiment of the invention, which is preferably dependent upon one of the preceding embodiments of the invention.

In a preferred embodiment of the method, in method steps f) and g), a plurality of, i.e., at least two, electrodes are provided and electrical contact is established therewith, wherein the plurality of electrodes are attached to be axially spaced apart from one another along a longitudinal axis of the catheter body. This embodiment is a tenth embodiment of the invention, which is preferably dependent upon one of the preceding embodiments of the invention.

In a preferred embodiment of the method, flushing passages are created axially between two axially adjacent electrodes in method step h). This embodiment is the eleventh embodiment of the invention, which is preferably dependent upon the tenth embodiment of the invention.

In a preferred embodiment of the method, the arrangement in method step c) involves helically winding the flexible circuit board around the inner liner. This embodiment is a twelfth embodiment of the invention, which preferably depends upon one of the preceding embodiments of the invention.

A thirteenth embodiment of the invention relates to a catheter body manufactured according to one of the first through twelfth embodiments of the invention, wherein the flushing passages have a diameter ranging from 100 µm to 500 mm.

In a preferred embodiment of the catheter body, a single inner lumen is formed within the inner liner. This embodiment is a fourteenth embodiment of the invention, which is preferably dependent upon the thirteenth embodiment of the invention.

In a preferred embodiment of the catheter body, the flushing passages are distributed radially around a circumference of the catheter body. This embodiment is a fifteenth embodiment of the invention, which is preferably dependent upon the thirteenth or fourteenth embodiment of the invention.

### IN GENERAL

In addition to the embodiments described herein, the elements of which “contain” or “comprise” a particular feature (e.g., a material), a further embodiment is always contemplated in which the element in question consists solely of the feature, i.e., does not comprise any other components. The word “comprise” or “comprising” is herein used synonymously with the word “contain” or “containing.”

When, in an embodiment, an element is referred to in the singular, an embodiment is also contemplated in which several of these elements are present. The use of a term for an element in the plural generally also includes an embodiment in which only a single corresponding element is included.

Unless otherwise stated or, from the context, clearly excluded, it is fundamentally possible and is hereby clearly considered that features of different embodiments can also be provided in the other embodiments described herein. It is also generally contemplated that all features described herein in connection with a method are also applicable to the products and devices described herein, and vice versa.

Merely for the sake of conciseness, these considered combinations are not all explicitly listed in all cases. Technical solutions that are known to be equivalent to the features described herein shall also be included in principle in the scope of the invention.

In the present description, range specifications also include the values specified as limits. An indication of the type “in the range of X to Y” with respect to a variable A consequently means that A can assume the values X, Y and values between X and Y. Ranges which are limited on one side, of the type "up to Y" for a size A, accordingly, mean a value Y and less than Y.

Some of the features described are associated with the term “substantially.” The term “substantially” is to be understood in such a way that, under real conditions and manufacturing techniques, a mathematically exact interpretation of terms such as “superimposition,” “perpendicular,” “diameter,” or “parallelism” can never be given exactly, but only within certain manufacturing error tolerances. For example, "substantially perpendicular axes" form an angle of 85 degrees to 95 degrees relative to one another, and "substantially equal volumes" comprise a variation of up to 5 vol.%.For example, a "device consisting substantially of plastic" comprises a plastic content of ≥95 to ≤100 wt.%.

For example, a " substantially complete filling of a volume B" comprises a filling of ≥95 to ≤100 vol.%. of the total volume of B.

The invention firstly relates to a method for producing a catheter body, in particular for an ablation catheter, comprising the method steps of:

a) providing a hollow-cylindrical inner liner with an inner lumen and a radially outer inner liner outer surface;

b) providing a flexible circuit board comprising a plurality of electrical conductors;

c) arranging the flexible circuit board on the inner liner outer surface;

d) sheathing the inner liner and the flexible circuit board with an outer insulation;

e) establishing a connection between the inner liner and the outer insulation so that the flexible circuit board is fixed between the inner liner and the outer insulation;

f) providing at least one electrode;

g) establishing electrical contact between the at least one electrode and at least one of the electrical conductors of the flexible circuit board; and,

h) creating a plurality of flushing passages which fluidically connect the inner lumen to a surface of the catheter body.

The method is used to produce a catheter body for a catheter, in particular for an ablation catheter. A catheter body is the elongated, tube-like portion of a catheter which, depending upon the application of the catheter, is equipped with a plurality of electrodes, at least at a distal catheter body end, preferably both at the distal catheter body end and at a proximal catheter body end that is axially opposite the distal catheter body end. The electrodes at the proximal end of the catheter body are sometimes called connectors.

In method step a), a hollow-cylindrical inner liner comprising a radially outer inner liner outer surface is provided. The inner liner extends axially from a proximal catheter body end to a distal catheter body end and radially surrounds at least one inner lumen extending axially through the inner liner, and thus also the final catheter body. The inner lumen serves at least for conducting a fluid, i.e., a liquid or a gas, which can be drained from the catheter body through the flushing passages when the catheter body is used. When using the catheter body, the fluid, e.g., a cooling liquid or a cooling gas, serves to cool the catheter and/or the body tissue surrounding the catheter.

The inner lumen, or at least part thereof, may further be used for the passage and/or introduction of various devices. For example, the inner lumen can also be used to insert a guide wire, which facilitates the insertion of the catheter into the patient. For this purpose, the inner lumen can be divided into several compartments by means of an inner wall or walls to create separate sub-regions - for example, for the fluid and the various devices. Such catheters with a divided inner lumen are often also referred to as multi-lumen catheters.

The inner liner has an inner liner inner diameter which is, for example, in a range of 0.75 to 4 mm, and an inner liner outer diameter which is, for example, in a range of 1 to 4.5 mm. Depending upon the application, the inner liner can have different lengths. For example, the inner liner has a length in the range of 30 cm to 200 cm.

The inner liner can be made of a variety of different materials, wherein the use of a flexible material or a flexible material combination is preferred. For example, the inner liner is made of a polymer that is selected from the group of silicones, polyolefins (e.g., polyethylene), polyether block amide (e.g., PEBAX®), polyurethanes, polyimides, polyamides, polyarylether ketones (e.g., polyether ether ketone), fluorinated polymers (e.g., selected from the group of ethylene tetrafluoroethylene, polytetrafluoroethylene, perfluoroalkoxyalkanes, polyvinylidene fluorides, fluorinated ethylene propylene, and mixtures thereof), and mixtures thereof. According to a preferred embodiment, the inner liner comprises a polymer that is selected from the group of silicones, polyolefins (e.g., polyethylene), polyether block amide (e.g., PEBAX®), polyurethanes, polyimides, polyamides, polyarylether ketone (e.g., polyether ether ketone), fluorinated polymers (e.g., selected from the group of ethylene tetrafluoroethylene, polytetrafluoroethylene, perfluoroalkoxyalkanes, polyvinylidene fluorides, fluorinated ethylene propylene, and mixtures thereof), and mixtures thereof.

In method step b), a flexible circuit board comprising a plurality of electrical conductors is provided. The flexible circuit board, sometimes also called flex circuit ribbon, is a circuit board comprising or consisting of flexible, bendable materials that allow the circuit board to be bent or folded or wound into different shapes. The structure of a typical flexible circuit board comprises several layers. The core element is one or more electrically conductive layers, e.g., made of a metal foil or a printed metal layer, preferably comprising or consisting of copper, which can contain a specific pattern, depending upon the type of application. This layer or a conductor track within this layer acts as the electrical conductor of the flexible circuit board. The electrically conductive layers are usually embedded between electrically insulating layers made of flexible insulation material such as polyimides, which can also provide mechanical protection.

The flexible circuit board of the method according to the invention comprises a plurality of, i.e., at least two, electrical conductors. For example, the flexible circuit board comprises two to 20 electrical conductors, each of which can be electrically connected to one or more electrodes. For example, the flexible circuit board comprises 12 electrical conductors which can be selectively connected to 12 electrodes of the catheter body. Preferably, the electrical conductors are electrically isolated from each other. More preferably, the flexible circuit board comprises at least two insulating layers which electrically insulate the at least one electrical conductor from the outside at least over long sections of the flexible circuit board. The flexible circuit board can have contact surfaces at certain positions which make the electrical conductor electrically contactable from the outside without the need to remove part of the insulating layer beforehand.

One advantage of using a flexible circuit board is its small space requirement, low weight, and simplified mounting. In particular, by using a flexible circuit board, there is no need to mount individual conductive wires or cables for establishing electrical contact with the electrodes of the catheter body, particularly through the inner lumen of the inner liner.

In method step c), the flexible circuit board is arranged on the inner liner outer surface. The flexible circuit board is preferably arranged in such a way that the flexible circuit board allows for an electrical connection between electrodes at the distal catheter body end of the catheter body and electrodes at the proximal catheter body end. After being arranged, the flexible circuit board thus extends along large parts of the inner liner, e.g., along at least 70%, preferably along at least 80%, more preferably along at least 90%, of the total length of the inner liner. In one embodiment, the flexible circuit board extends substantially over the entire length of the inner liner. Arranging the flexible circuit board on the inner liner outer surface may involve simply placing it on the inner liner such that the longitudinal axes of the inner liner and the flexible circuit board are substantially parallel with one another, for example. To simplify the arrangement thereof and the subsequent method steps, in particular method step d), it may be advantageous to fix the flexible circuit board to the inner liner outer surface, at least temporarily, e.g., by means of an adhesive such as a glue or an adhesive tape - for example, by means of a double-sided adhesive tape.

In method step d), the inner liner and the flexible circuit board arranged on the outer surface of the inner liner are sheathed, in particular coaxially sheathed, or at least partially sheathed, with outer insulation. In one embodiment, the sheathing process is carried out by sliding, or in other words “pulling," hollow-cylindrical outer insulation onto the inner liner. In this embodiment, the outer insulation is preferably in the form of a hollow-cylindrical hose that has an inner insulation diameter which substantially corresponds to the inner liner outer diameter. The insulation inner diameter and/or the material of the outer insulation must be selected so that the flexible circuit board can be arranged between the inner liner and the outer insulation. In a further embodiment, the sheathing process is carried out by extruding, in particular coaxially extruding, outer insulation around the inner liner and the flexible circuit board.

By sheathing the inner liner, it is coaxially surrounded by the outer insulation, wherein the inner liner outer surface faces an insulation inner surface. The outer insulation is preferably the radially outer layer of the catheter and serves in particular to electrically insulate the catheter body from the outside and to mechanically protect the catheter body. Preferably, the outer insulation has an outer diameter in a range of 200 µm to 5,000 µm, more preferably 300 µm to 3,000 µm and most preferably 500 µm to 1,500 µm. In an exemplary embodiment, the outer diameter of the outer insulation is about 700 µm. It goes without saying that the outer diameter of the outer insulation can also correspond to the outer diameter of the catheter body. The outer insulation preferably has a wall thickness in the range of 2 µm to 300 µm, particularly preferably from 5 µm to 150 µm, and most preferably from 20 µm to 100 µm. According to a preferred embodiment of the present invention, the outer insulation has an outer diameter in a range of 300 µm to 3,000 µm, particularly preferably 500 µm to 1,500 µm, and a wall thickness in a range of 5 µm to 150 µm, particularly preferably 20 µm to 100 µm. In an exemplary embodiment, the wall thickness is approximately 60 µm. The outer insulation preferably consists of a polymer that is selected from the group of silicones, polyolefins (e.g., polyethylene), polyether block amide (e.g., PEBAX®), polyurethanes, polyimides, polyamides, polyarylether ketones (e.g., polyether ether ketone), fluorinated polymers (e.g., selected from the group of ethylene tetrafluoroethylene, polytetrafluoroethylene, perfluoroalkoxyalkanes, polyvinylidene fluorides, fluorinated ethylene propylene, and mixtures thereof), and mixtures thereof. According to a preferred embodiment, the outer insulation comprises a polymer that is selected from the group of silicones, polyolefins (e.g., polyethylene), polyurethanes, polyimides, polyamides, polyarylether ketone (e.g., polyether ether ketone), fluorinated polymers (e.g., selected from the group of ethylene tetrafluoroethylene, polytetrafluoroethylene, perfluoroalkoxyalkanes, polyvinylidene fluorides, fluorinated ethylene propylene, and mixtures thereof), and mixtures thereof, and has an outer diameter in the range of 300 to 3,000 µm.

The flexible circuit board is arranged radially between the inner liner and the outer insulation, i.e., between the insulation inner surface and the inner liner outer surface.

In method step e), a connection is established between the inner liner, in particular the inner liner outer surface, and the outer insulation, in particular the insulation inner surface, so that the flexible circuit board, which is arranged radially between the inner liner and the outer insulation, is fixed between the inner liner and the outer insulation. Fixing the flexible circuit board in this way substantially prevents free movement of the flexible circuit board relative to the inner liner and/or the outer insulation. The location and orientation, or in other words the position, of the flexible circuit board within the catheter body is thus fixed by creating the connection between the inner liner and the outer insulation and substantially no longer changes, which represents an advantage over the wires or cables for creating electrical contact with the electrodes of the catheter body that are normally arranged in the inner lumen in the prior art. The fixed flexible circuit board, whose position and orientation is either known or can be easily determined, e.g., by using transparent outer insulation or by applying markings to the outer surface of the catheter body, can significantly simplify the further method steps. Depending upon the design of the method, the position and orientation of the flexible circuit board can also be stored electronically - for example, in a CAT file. In particular, the flexible circuit board fixed in this way allows for the subsequent creation of flushing passages without the risk of unwanted severing of the electrical conductors of the flexible circuit board.

In method step f), at least one electrode is provided. The exact number of electrodes depends upon what the catheter is being used for. Preferably, as many electrodes as electrical conductors are provided so that each of the electrodes can be electrically connected to an electrical conductor during the method.

Preferably, the electrodes are ring electrodes. Further preferably, all provided electrodes are ring electrodes.

The electrode can comprise a plurality of different materials or consist of different materials. Preferably, the electrode comprises a metal that is selected from the group consisting of platinum, iridium, tantalum, palladium, titanium, iron, gold, molybdenum, niobium, tungsten, nickel, chromium, cobalt, steel, nitinol, alloys of any of these metals, and composites of any of these metals. Stainless steel is suitable as an electrode - for example, stainless steel AISI 316L, stainless steel AISI 301, or stainless steel AISI 304. Platinum and platinum alloys such as Pt/Ir 10 or Pt/Ir 20 or nickel-cobalt alloys such as MP35N are also suitable as electrodes.

The choice of metal for the electrodes (and for the electrical conductors) can depend upon the use of the catheter according to the invention. However, it should be understood that the application of the catheter is not limited by the use of a particular metal.

The electrode can also have a coating. Suitable coatings are metal nitrides such as TiN, metal oxides such as IrO₂, or conductive polymers. The surface of the electrode can also be surface-structured, e.g., laser-structured.

The electrode, preferably a ring electrode, can have an outer diameter in the range of 200 to 5,000 µm, in one embodiment in the range of 300 to 3,000 µm, and in one embodiment in the range of 500 to 1,500 µm. The electrode can have a wall thickness in the range of 10 to 200 µm, in one embodiment 10 to 100 µm, and in one embodiment 30 to 70 µm. Furthermore, the electrode can have a length in the range of 200 to 5,000 µm, in one embodiment 300 to 3,000 µm, and in one embodiment in the range of 500 to 1,500 µm. According to one embodiment, the electrode has an outer diameter in the range of 300 to 3,000 µm and in one embodiment in the range of 500 to 1,500 µm, a wall thickness in the range of 10 to 100 µm and in one embodiment of 30 to 70 µm, and a length in the range of 300 to 3,000 µm and in one embodiment in the range of 500 to 1,500 µm.

In method step g), electrical contact is established between the at least one electrode, preferably all the electrodes, and at least one of the electrical conductors, preferably each of the electrodes and one, preferably exactly one, electrical conductor, of the flexible circuit board. Depending upon the design, establishing the electrical contact may involve removing parts of the insulation of the flexible circuit board so that the electrical conductor or the corresponding electrical conductors is/are available for establishing electrical contact with the electrode. Establishing the electrical contact may also involve mechanically fixing the electrode to the catheter body. In one embodiment, the at least one electrode is brought into direct physical contact with the at least one electrical conductor in order to create the electrical contact. In a further embodiment, the at least one electrode and the at least one electrical conductor are not electrically connected to one another via direct physical contact, but indirectly via an electrically conductive bridge element. Examples of bridge elements include metal elements such as metal plates, metal spring elements, or metal balls, as well as conductive adhesives, conductive pastes, or solders.

In method step g), a plurality of, i.e., at least two, flushing passages are created, which fluidically connect the inner lumen, or at least part of the inner lumen, e.g., a single compartment of the inner lumen, to the surface of the catheter body. The exact number of flushing passages and their position can be dependent upon the intended use of the catheter body. For example, the catheter body can comprise 2-200 flushing passages. The type of surface of the catheter body that is provided with flushing passages is dependent upon the location of the corresponding flushing passage. For example, the flushing passage can lead from the inner lumen into the region surrounding the catheter body via the inner liner and one of the electrodes, or the flushing passage can lead into the region surrounding the catheter body via the inner liner and the outer insulation. The flushing passage forms a fluidic connection, i.e., a type of tunnel, which extends radially outward from the inner lumen, or part of the inner lumen, through the wall of the catheter body, i.e., the inner liner, the outer insulation, and/or the at least one electrode.

Method steps a) to h) of the method according to the invention can, if technically possible and reasonable, be carried out in a different sequence and/or at least partly simultaneously.

A preferred embodiment of the method is characterized in that method step e) is carried out before method steps f) and g). In this embodiment, the flexible circuit board is fixed before the at least one electrode is mounted. Since, by fixing the flexible circuit board, the position of the electrical conductors is known, e.g., electronically recorded, e.g., by means of a CAT file, or is easily perceptible, e.g., by using transparent outer insulation, and essentially no longer changes, this sequence can simplify the process of establishing electrical contact with the at least one electrode. Furthermore, this sequence increases the flexibility of the method, since an otherwise identical catheter body can be easily manufactured in different positions with electrodes and with a different number of electrodes.

In one embodiment, method steps e) and g) take place simultaneously. In this embodiment, it is preferred that the process of sheathing the inner liner in method step d) involve partially sheathing the inner liner and the flexible circuit board, which is done by sliding on hollow-cylindrical outer insulation. Before method step e) is then carried out, the at least one electrode, preferably in the form of a ring electrode, is also slid onto the inner liner to the desired position and orientation relative to the flexible circuit board. This process can be repeated with several pieces of outer insulation and electrodes, which are preferably each alternately slid onto the inner liner. Method steps e) and g) then take place simultaneously, preferably by heating. Furthermore, it may be advantageous to establish the electrical contact with the at least one electrode via an electrically conductive bridge element - for example, in the form of a solder paste or a conductive adhesive.

A preferred embodiment of the method is characterized in that method step e) is carried out before method step h). In this embodiment, the flexible circuit board is thus fixed before the plurality of flushing passages are created. Since, by fixing the flexible circuit board, the position of the electrical conductors is known, e.g., electronically recorded, e.g., by means of a CAT file, or easily perceptible, e.g., by using transparent outer insulation, and also substantially no longer changes, this sequence can facilitate the creation of the plurality of flushing passages. Furthermore, this sequence increases the flexibility of the method, since an otherwise identical catheter body with flushing passages and with a different number of flushing passages in different positions can be manufactured.

A preferred embodiment of the method is characterized in that method step e) is carried out before method steps f) and g), and method step h) is carried out after method steps f) and g). This embodiment of the method combines the above-described advantages of the specified sequence of method steps.

The electrical contact with the at least one electrode in method step g) can be established in different ways.

A preferred embodiment of the method is characterized in that, for the establishment of electrical contact with the electrode, preferably all of the electrodes, in method step g), at least one contact opening is created in the outer insulation in order to selectively establish electrical contact between the electrode and at least one electrical conductor of the flexible circuit board that is accessible via the contact opening.

To create the contact opening, part of the outer insulation and, if necessary, part of the insulating layer of the flexible circuit board is removed at the corresponding position so that the at least one electrical conductor is exposed radially from the outside via the contact opening to be electrically contactable. The removal of parts of the insulating layer of the flexible circuit board is necessary if the flexible circuit board does not already comprise an accessible electrical conductor at the position of the contact opening or has a contact surface via which the electrical conductor can be electrically contacted from the outside without removing parts of the insulating layer. At the position of the contact opening, an electrode can be selectively, or in other words specifically, electrically contacted with the electrical conductor over the course of the subsequent method. If the catheter body is to have more than one electrode, in particular at the distal catheter body end, it is preferred that a separate contact opening be created for each of these electrodes, wherein one of the electrical conductors of the flexible circuit board is selectively electrically contacted via each of these contact openings. For this purpose, the flexible circuit board preferably has at least exactly as many electrical conductors as electrodes that are to be attached to the distal catheter body end.

The contact opening can be created in different ways. For example, the contact opening can be created by cutting, punching, or, preferably, by laser ablation.

The connection between the inner liner and the outer insulation in method step e) can be established in different ways.

For example, the inner liner and the outer insulation can be mechanically connected to one another - for example, by means of a clamp, a screw, a nail, or another type of positive and/or non-positive connection.

In another embodiment, the inner liner and the outer insulation may be glued together to establish the connection. Gluing can be done, for example, using an adhesive or an adhesive tape, such as double-sided adhesive tape. For example, the inner liner outer surface and/or the insulation inner surface can be coated with an adhesive layer, which forms the connection when the two elements are pressed against one another, for example.

A preferred embodiment of the method is characterized in that, in order to establish the connection between the inner liner and the outer insulation in method step e), the inner liner and/or the outer insulation are warmed up, or in other words “heated," forming an integral bond between the inner liner and the outer insulation. In this embodiment, the inner liner and the outer insulation “fuse” together, at least at their adjoining outer surfaces, i.e., the inner liner outer surface and the insulation inner surface. It goes without saying that neither the inner liner nor the outer insulation need to be completely converted to a liquid state for this purpose, but, rather, it is preferable to simply cause the interface to “melt."

The plurality of flushing passages can be created in method step h) in different ways. For example, the flushing passages can be produced by cutting, drilling, or punching, wherein, of the three exemplary procedures listed, drilling is preferred for producing flushing passages which pass at least partially through one of the electrodes of the catheter body.

A preferred embodiment of the method is characterized in that the plurality of flushing passages are produced in method step h) by means of a laser. This is preferred because it is a cost-effective, fast, and flexible way of creating the flushing passages. A laser also makes it possible to create the flushing passages at any desired position, and thus both through the at least one electrode and next to the at least one electrode. In order to prevent the laser from undesirably simultaneously creating another flushing passage in the opposite wall after creating a flushing passage in one wall of the catheter body and the laser thus not “shooting through” the catheter body, it may be preferable for a preferably metal mandrel to be arranged in the inner lumen, which mandrel blocks the laser from striking the opposite wall after creating the desired flushing passage. The mandrel can further simplify the method as a whole, but in particular method steps c) and d), such that it is preferred to provide the inner liner from method step a) pulled onto a mandrel.

The flushing passages can be created at any position on the catheter body.

A preferred embodiment of the method is characterized in that, in method step h), flushing passages, i.e., all the flushing passages or only some of the flushing passages, are produced, which extend at least through the at least one electrode and the inner liner. In this embodiment, at least some of the flushing passages open into a surface of the at least one electrode.

These flushing passages thus form a direct fluidic connection between the inner lumen and the surface of the corresponding electrode, which can simplify cooling of the corresponding electrode and the adjacent body tissue when using the catheter body.

A preferred embodiment of the method is characterized in that, in method step h), flushing passages, i.e., all the flushing passages or only some of the flushing passages, are produced, which extend at least through the outer insulation and the inner liner. In this embodiment, the flushing passages, or at least some of the flushing passages, do not extend through the electrodes of the catheter body. The flushing passages are thus arranged only adjacently to the electrodes and preferably open into the surface of the outer insulation. These flushing passages thus form a direct fluidic connection between the inner lumen and the surface of the outer insulation.

This may be preferred because such flushing passages, due to the preferred materials of the inner liner and the outer insulation, are easier to produce than flushing passages that also extend through the electrodes.

A preferred embodiment of the method is characterized in that, in method steps f) and g), a plurality of electrodes are provided and electrical contact is established therewith, wherein the plurality of electrodes are attached so as to be axially spaced apart from one another along a longitudinal axis of the catheter body. Preferably, a portion of the outer insulation is attached to the corresponding portion of the inner liner between each two axially adjacent electrodes.

A preferred embodiment of the method is characterized in that, in method step h), flushing passages, i.e., all the flushing passages or only some of the flushing passages, are created axially between two axially adjacent electrodes. Preferably, these flushing passages are not formed through the axially adjacent electrodes, but through the portions of the inner liner and the outer insulation between the axially adjacent electrodes.

The flushing passages can also be created through the flexible circuit board, in particular through regions of the flexible circuit board which have no electrical conductors. To minimize the risk of unintentional damage to an electrical conductor of the flexible circuit board during the creation of the flushing passages, it is preferred that the flushing passages be created, not through the flexible circuit board, but only adjacent to the flexible circuit board.

As already mentioned, the flexible circuit board can be arranged in method step c) in different ways - for example, by also at least temporarily fastening the flexible circuit board to the inner liner outer surface. The fastening can be achieved using an adhesive such as a glue or an adhesive tape. Furthermore, the flexible circuit board can have different orientations on the inner liner outer surface. For example, the flexible circuit board can be arranged substantially along the longitudinal axis of the inner liner. In this embodiment, the longitudinal axes of the flexible circuit board and the inner liner are substantially parallel to one another. This is a particularly simple type of arrangement.

A preferred embodiment of the method is characterized in that the arrangement of the circuit board in method step c) involves helically winding the flexible circuit board around the inner liner, and thus on the inner liner outer surface. In this embodiment, the flexible circuit board is thus arranged axially in helical windings, preferably along substantially the entire length of the inner liner. The helical arrangement of the flexible circuit board can give the finished catheter body particular resistance to kinking or breaking of the electrical conductors of the flexible circuit board, since the windings can allow for improved bending thereof. Furthermore, the helical arrangement provides greater flexibility when positioning the flushing passages, since the flexible circuit board does not continuously cover the same side of the inner liner and can therefore block for the creation of the flushing passages in a subsequent method step. Furthermore, the arrangement of the circuit board in windings increases the flexibility of the electrical contact with the electrodes of the catheter body, which therefore does not always have to take place on the same side of the inner liner. Especially with electrodes that are not ring electrodes and therefore do not extend around the entire circumference of the catheter body, the flexibility of the method is increased regarding positioning of the electrodes.

The number of windings and the distance between two adjacent windings can depend upon various factors, such as the width of the flexible circuit board, the number of electrical conductors, and the intended number, position, and size of the flushing passages.

The invention also relates to a catheter body produced according to the method described above, which is characterized in that the flushing passages have a diameter ranging from 100 µm to 500 µm. Such diameters can be easily realized using the method described above and allow for the sufficient conduction of the desired fluid, such as a cooling liquid, from the inner lumen into the region around the catheter body.

The inner lumen of the catheter body may comprise a plurality of, i.e., at least two, separate compartments extending axially through the catheter body. The individual compartments can be fluidically separated from one another by internal walls. Catheters comprising such a catheter body are often called multi-lumen catheters.

The production of such a catheter body is preferably carried out by providing an inner liner with the desired number of separate compartments in method step a).

A preferred embodiment of the catheter body is characterized in that a single inner lumen is formed within the inner liner. Catheters with such a catheter body are often referred to as open-lumen catheters. Such a catheter body has the advantage that the flushing passages can be easily created anywhere around the circumference of the catheter body, increasing the flexibility of the design of the catheter body as well as its manufacturing method.

A preferred embodiment of the catheter body is characterized in that the flushing passages, i.e., all the flushing passages or only some of the flushing passages, are distributed radially around a circumference of the catheter body. Such a catheter body has the advantage that the desired fluid can be discharged from the inner lumen in all radial directions, which allows for its uniform distribution.

The properties and features disclosed in the description may be essential for various embodiments of the invention, both separately and in any combination with one another. The properties and features disclosed for the method are also disclosed for the catheter body, and vice versa.

### BRIEF DESCRIPTION OF THE DRAWINGS

The invention is further illustrated by way of example below by means of figures. The invention is not limited to the figures.

### In the figures:

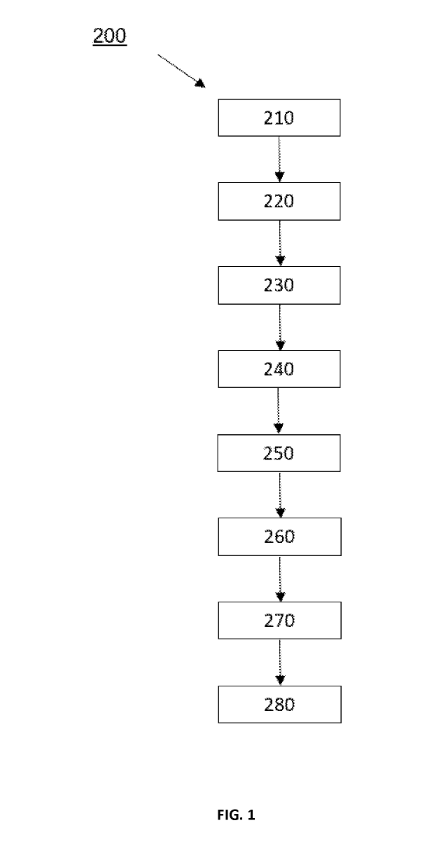

FIG. 1 is an exemplary flowchart of a method for producing a catheter body;

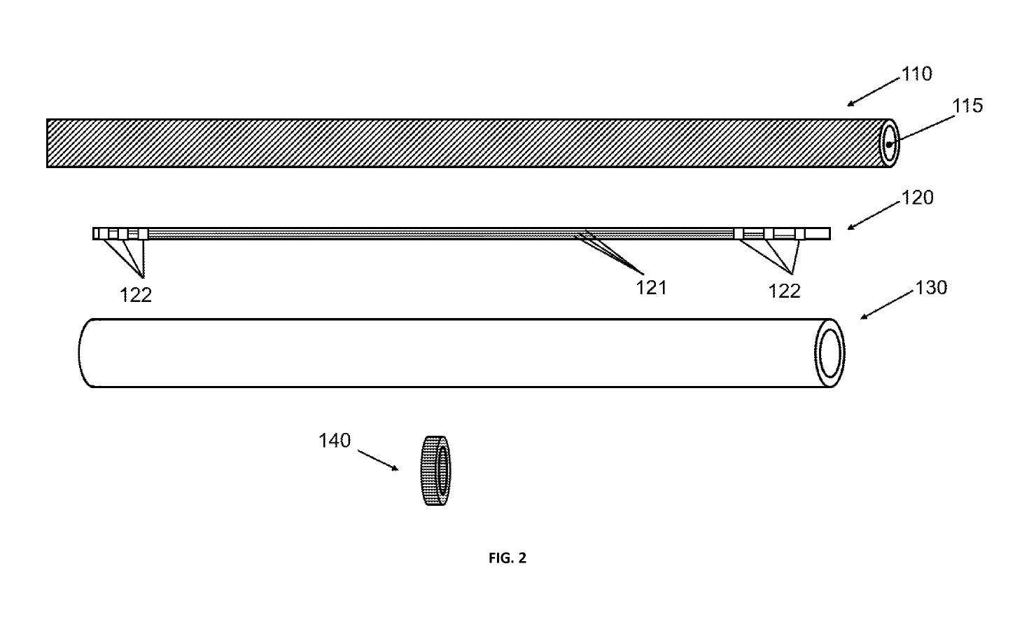

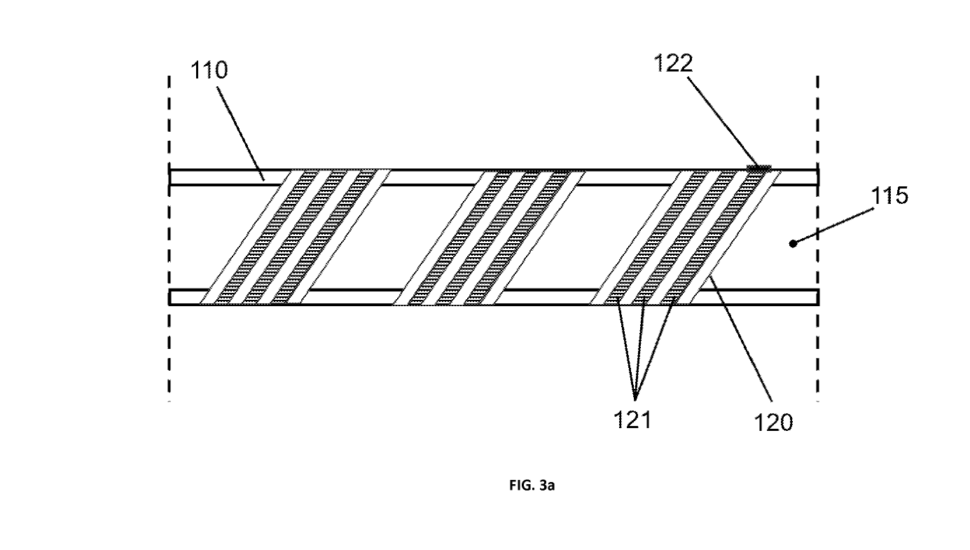

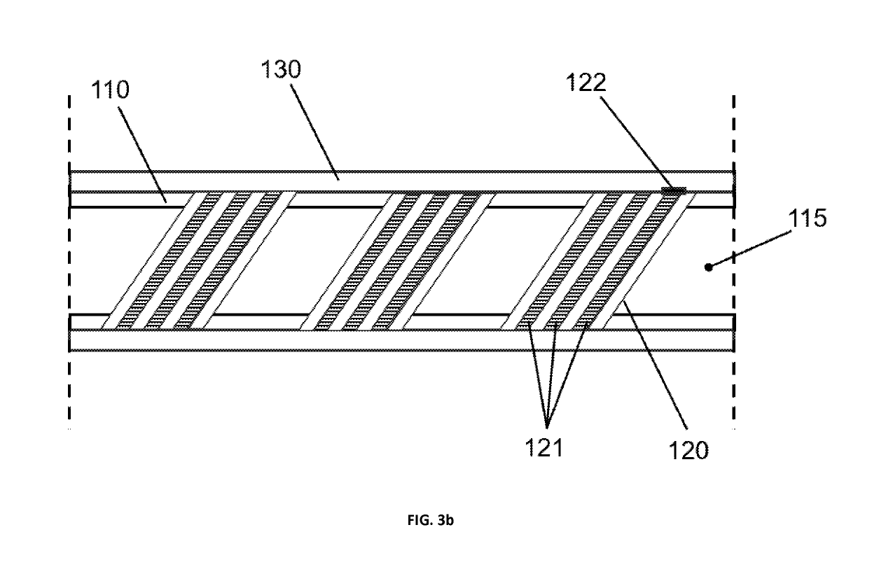

FIG. 2 shows exemplary schematic components for use in a method for producing a catheter body, in a perspectival side view;

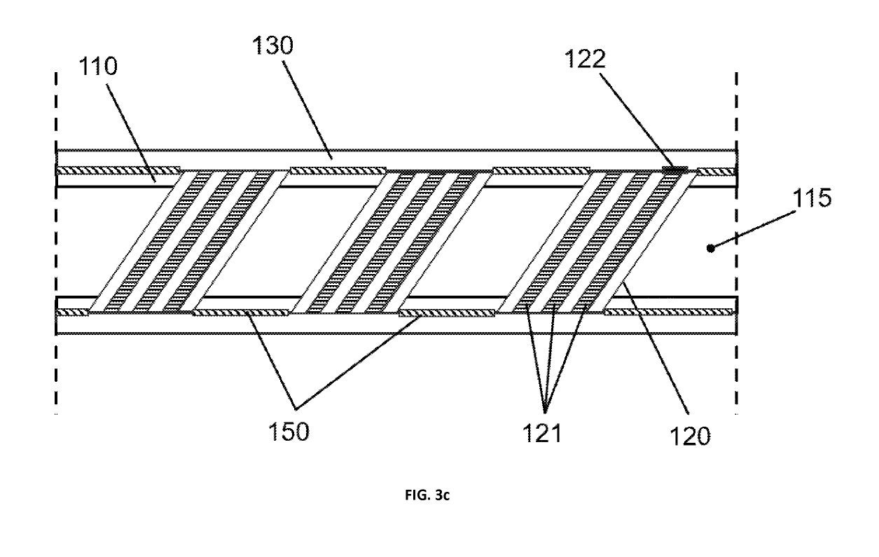

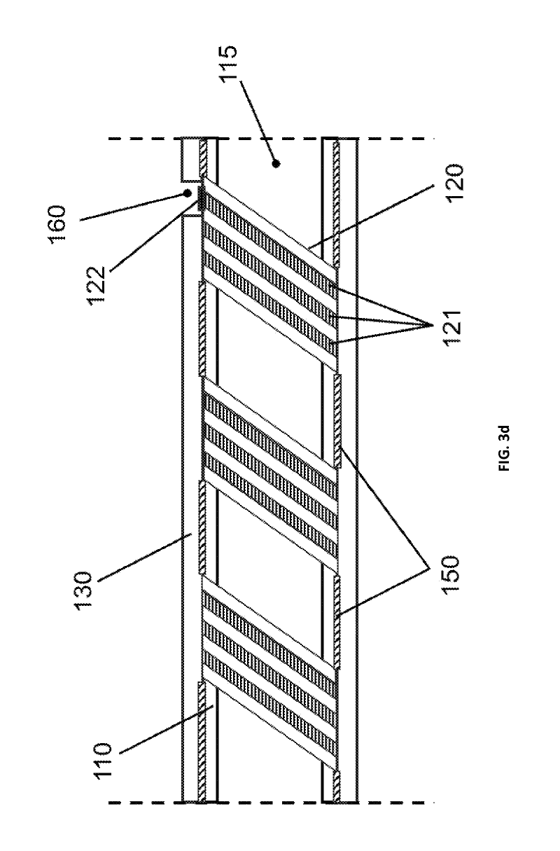

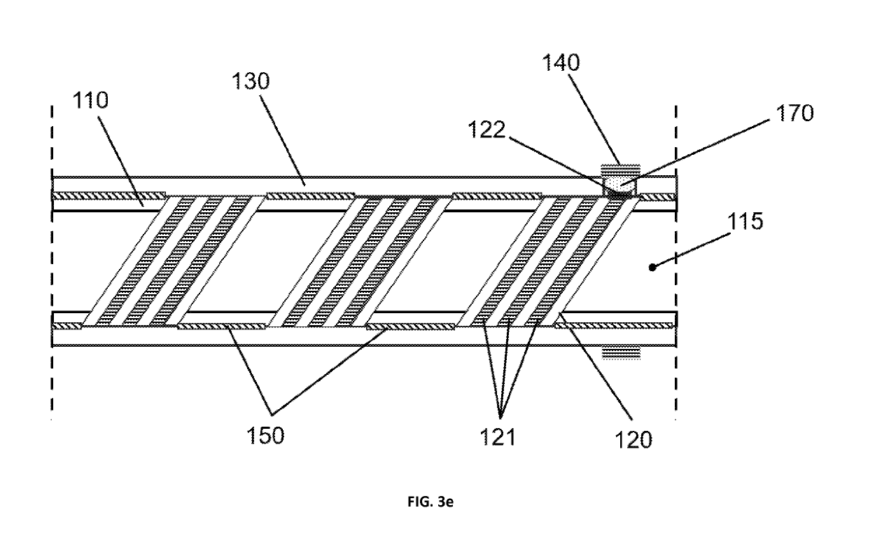

FIGS. 3a-e are schematic longitudinal sections of exemplary method steps or intermediate products 3a to 3e of the method from FIG. 1 shown with the components from FIG. 2; and,

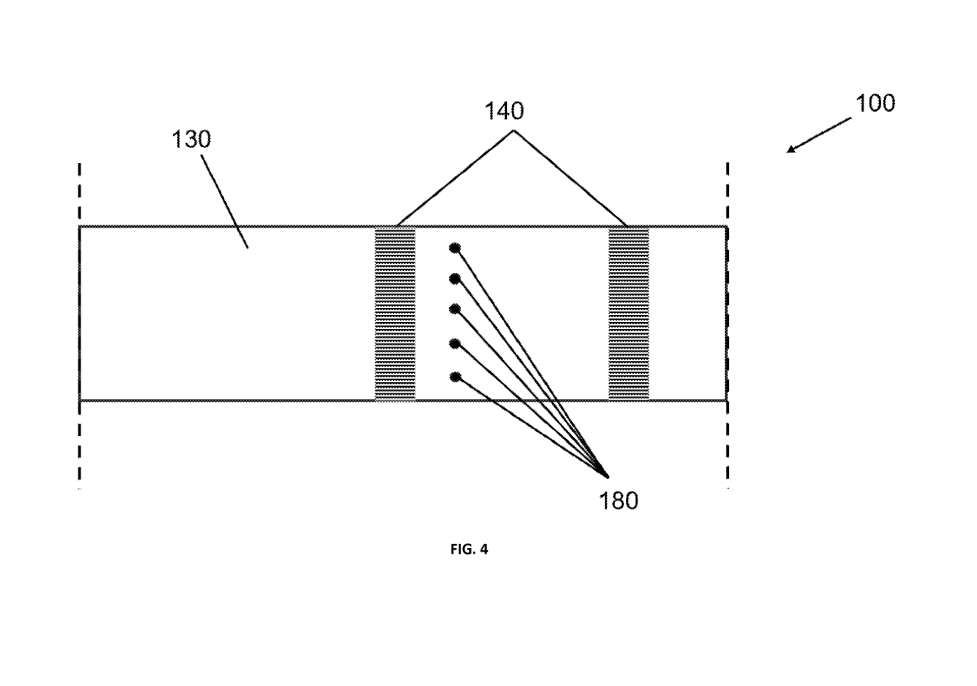

FIG. 4 is a schematic external view of an exemplary catheter body.

### DETAILED DESCRIPTION OF THE INVENTION

FIG. 1 shows a flowchart of an exemplary method 200 for producing a catheter body 100 comprising the method steps 210 to 280.

In method step 210, a hollow-cylindrical inner liner 110 with an inner lumen 115 and a radially outer inner liner outer surface is provided.

In method step 220, a flexible circuit board 120 comprising a plurality of electrical conductors 121 is provided. The electrical conductors 121 are used to electrically connect electrodes 140, preferably at a distal end of the catheter body 100, to further electrodes, often also referred to as connectors, or other devices and systems at a proximal end, opposite the distal end, of the catheter body 100. The flexible circuit board 120, or its electrical conductors 121, thus extend(s) over large parts, e.g., over at least 70%, of the total length of the final catheter body 100.

In method step 230, the flexible circuit board 120 is arranged on the inner liner 110, in particular the inner liner outer surface. The flexible circuit board can be arranged in different ways, wherein it is preferred that the flexible circuit board 120 be helically wound around the inner liner 110. The arrangement of the flexible circuit board can be supported, at least temporarily, by an adhesive, e.g., a glue or an adhesive tape, particularly a double-sided adhesive tape.

In method step 240, the inner liner 110 and the flexible circuit board 120 arranged on the inner liner 110 are sheathed with outer insulation 130. This can be done, for example, by coaxially extruding, or in other words “extruding-on," a polymer, for example, and the inner liner. Preferably, the sheathing process is carried out by pulling hollow-cylindrical outer insulation 130 onto the inner liner 110 and the flexible circuit board 120 arranged on the inner liner 110.

In method step 250, a connection 150 is established between the inner liner 110 and the outer insulation 130 so that the flexible circuit board 120 is fixed between the inner liner 110 and the outer insulation 130. By means of the connection 150 of the inner liner 110 and the outer insulation 130, the position of the flexible circuit board 120 is substantially unchangeable, thus simplifying the further steps of the method 200.

In method step 260, at least one electrode 140 is provided. Preferably, the electrode 140 comprises a metal. More preferably, the electrode 140 is a ring electrode, preferably a metallic ring electrode.

In method step 270, electrical contact is established between the at least one electrode 140 and at least one electrical conductor 121, preferably with exactly one of the electrical conductors 121, of the flexible circuit board 120. Preferably, establishing electrical contact involves creating at least one contact opening 160 near a distal catheter body end. The contact opening 160 serves to expose the at least one electrical conductor 121 of the flexible circuit board 120 so that electrical contact can be established therewith from outside the catheter body 100. To do this, at least parts of the outer insulation 130 above the position of the electrical conductor 121 that is to be contacted are removed. If the electrical conductor 121 is covered by one or more insulating layers of the flexible circuit board 120 in this position, the creation of the contact opening 160 also involves the removal of this/these insulating layer(s). The contact opening 160 is preferably created by laser ablation. Preferably, the electrical conductor 121 comprises a contact surface 122 at the corresponding position, which surface can be freely electrically contacted from outside the flexible circuit board so that the (partial) removal of insulating layers of the flexible circuit board 120 is not necessary. Establishing the electrical contact may also involve the use of an electrically conductive bridge element 170, such as a solder or a conductive adhesive, which is applied between the electrical conductor 121 and the electrode 140.

In method step 280, a plurality of flushing passages 180 are created, which fluidically connect the inner lumen 115 of the inner liner 110 to a surface of the catheter body 110. The flushing passages 180 can, for example, open at the surface into one of the electrodes 140 or into the outer insulation 130. Preferably, the flushing passages 180 are created by laser ablation.

Method steps 210 to 280 can be carried out in any sequence, provided this is technically possible and reasonable. Preferably, at least method step 280 is carried out after method step 250. This simplifies method step 280 and increases the flexibility of the method 200.

FIG. 2 is a schematic, perspectival side view of various exemplary components of the method 200 for producing a catheter body 100. Shown are a hollow-cylindrical inner liner 110 with an inner lumen 115, hollow-cylindrical outer insulation 130, an electrode 140 in the form of a metallic ring electrode, as well as a flexible circuit board 120. The flexible circuit board 120 has three electrical conductors 121, each extending from an axial end of the flexible circuit board 120 to an opposite axial end of the flexible circuit board 120. At both ends of the flexible circuit board 120, the electrical conductors 121 selectively terminate in each case in three contact surfaces 122 which are designed to selectively electrically contact one of the electrical conductors 121 at the distal end and/or at the proximal end of the flexible circuit board 120. In other words, each of the electrical conductors 121 extends from one of the contact surfaces 122 at one end of the flexible conductor 120 to one of the contact surfaces 122 at the opposite end of the flexible conductor 120. In the generally preferred embodiment of the flexible circuit board 120 shown, the electrical conductors 121, except for the contact surfaces 122 at both ends thereof, are electrically insulated from the outside, preferably by being embedded in one or more electrically insulating layers. The contact surfaces 122, on the other hand, can be electrically contacted from the outside and are therefore freely accessible. The inner and outer diameters of the inner liner 110 and the outer insulation 130 correspond to one another so that the outer insulation 130 can be pushed coaxially onto the inner liner 110.

FIGS. 3a-e show various intermediate products or method steps of the method 200 from FIG. 2 on the basis of the components of FIG. 2.

FIG. 3a shows a portion of the inner liner 110 around which the flexible circuit board 120 is helically wound, such that it is arranged on an inner liner outer surface of the inner liner 110. For better clarity, the flexible circuit board 120 is shown in a top view. One of the electrical conductors 121 has one of its contact surfaces 122 in the section shown.

FIG. 3b shows the inner liner 110 and the flexible circuit board 120 from FIG. 3a, both of which have been sheathed with the outer insulation 130 by pulling it on. The flexible circuit board 120 is thus arranged radially between the inner liner 110 and the outer insulation 130 - more precisely, between the inner liner outer surface and a radially inner insulation inner surface.

FIG. 3c shows the inner liner 110, the outer insulation, and the flexible circuit board 120, arranged between the inner liner 110 and the outer insulation 130, from FIG. 3b, wherein a connection 150 (indicated by striped regions between the inner liner 110 and the outer insulation 130; provided with reference signs only by way of example) - this being an integral bond 150 in the embodiment shown - has been created between the inner liner 110 and the outer insulation 130 at the points where the two adjoin one another. In the embodiment shown, the connection 150 between the inner liner 110 and the outer insulation 130 was established by heating them, such that they fused together. The connection 150 fixes the flexible circuit board 120 between the inner liner 110 and the outer insulation 130, so that its position essentially no longer changes for the subsequent method steps. In the embodiment shown, the outer insulation 130 consists of a transparent polymer, so that the position of the flexible circuit board 120 can be optically determined at any time. In further embodiments (not shown), the position of the flexible circuit board 120 is stored electronically, e.g., in a CAT file, or the position is noted by one or more markings - for example, on the outside of the outer insulation 130.

FIG. 3d shows the arrangement from FIG. 3c, wherein a contact opening 160 has been created in the outer insulation 130, through which one of the electrical conductors 121 of the flexible circuit board 120 can be electrically contacted via its contact surface 122 that is accessible from the outside via the contact opening 160. In the embodiment shown, the contact surface 160 was created by laser ablation.

FIG. 3e shows the arrangement from FIG. 3d, wherein a bridge element 170 in the form of a conductive adhesive, e.g., comprising a silver powder dispersed in a curable organic medium, has been introduced into the contact opening 160 (see FIG. 3d). After the bridge element 170 was inserted, the ring electrode 140 was pushed onto the arrangement at the axial height of the contact opening 160 and to be in contact with the bridge element 170. By curing the bridge element 170, electrical contact is established between the ring electrode and the corresponding electrical conductor 121 - indirectly via the bridge element 170 in the embodiment shown.

FIG. 4 is a schematic side view of the finished catheter body 100 from the one in FIG. 3c, wherein said catheter body is finished with a plurality of flushing passages 180 (only the flushing passages on the side shown in the side view are visible) which fluidically connect the inner lumen 115 (see FIGS. 2 and 3a-e) and a surface of the catheter body 100. In the embodiment shown, the flushing passages 180 extend beyond the inner liner 110 and the outer insulation 130 from the inner lumen 115 to the radially outer surface of the outer insulation 130, which also simultaneously forms the surface of the catheter body 100 in the corresponding position. The flushing passages 180 are arranged such that they do not extend through the flexible circuit board 130. In the embodiment shown, the flushing passages 180 have been created by means of a laser, wherein, for this purpose or already in one of the preceding method steps, a mandrel (not shown), preferably made of a metal, can be introduced into the inner lumen 115, so that the laser penetrates only one wall of the catheter body 100 to create a flushing passage 180 without also penetrating the opposite wall of the catheter body 100 at the same time. The flushing passages 180 allow a fluid, such as a cooling liquid, to be discharged from the inner lumen 115 onto the surface of the catheter body 100, where, depending upon the region around the catheter body 100 and the nature of the fluid, it can be further distributed and can serve, for example, to cool the catheter body, in particular the electrodes 140, and/or the body tissue of a patient surrounding the catheter body 100. Previously fixing the flexible circuit board 120 (see FIGS. 3c-e)facilitates the creation of the flushing passages 180, since the risk of damage to the electrical conductors 121, which substantially no longer move out of their position after the flexible circuit board 120 has been fixed, is reduced.

### REFERENCE SIGNS

100 Catheter body

110 Inner liner

115 Inner lumen

120 Flexible circuit board

121 Electrical conductor

122 Contact surface

130 Outer insulation

140 Electrode

150 Connection

160 Contact opening

170 Bridge element

180 Flushing passage

200 Method for producing a catheter body

210 Providing an inner liner

220 Providing a flexible circuit board

230 Arranging the flexible circuit board

240 Sheathing the inner liner and the flexible circuit board

250 Establishing a connection

260 Providing an electrode

270 Establishing electrical contact with the electrode

280 Creating flushing passages

What is claimed is:

1. A method for producing a catheter body comprising flushing passages, comprising the method steps of:
a) providing a hollow-cylindrical inner liner with an inner lumen and a radially outer inner liner outer surface;
b) providing a flexible circuit board comprising a plurality of electrical conductors;
c) arranging the flexible circuit board on the inner liner outer surface;
d) sheathing the inner liner and the flexible circuit board with an outer insulation;
e) establishing a connection between the inner liner and the outer insulation so that the flexible circuit board is fixed between the inner liner and the outer insulation;
f) providing at least one electrode;
g) establishing electrical contact between the at least one electrode and at least one of the electrical conductors of the flexible circuit board;
h) creating a plurality of flushing passages which fluidically connect the inner lumen to a surface of the catheter body.

2. The method according to claim 1, wherein method step e) is carried out before method steps f) and g).

3. The method according to claim 1, wherein method step e) is carried out before method step h).

4. The method according to claim 1, wherein method step e) is carried out before method steps f) and g), and method step h) is carried out after method steps f) and g).

5. The method according to claim 1, wherein for establishing electrical contact with the electrode in method step g), at least one contact opening is created in the outer insulation in order to selectively establish electrical contact between the electrode and at least one electrical conductor of the flexible circuit board that is accessible via the contact opening.

6. The method according to claim 1, wherein for establishing the connection between the inner liner and the outer insulation in method step e), the inner liner and the outer insulation are heated, forming an integral bond between the inner liner and the outer insulation.

7. The method according to claim 1, wherein a laser is used for the creation of the plurality of flushing passages in method step h).

8. The method according to claim 1, wherein flushing passages are created in method step h) which extend at least through the at least one electrode and the inner liner.

9. The method according to claim 1, wherein flushing passages are created in method step h) which extend at least through the outer insulation and the inner liner.

10. The method according to claim 1, wherein in method steps f) and g), a plurality of electrodes are provided and electrical contact is established therewith, wherein the plurality of electrodes are attached so as to be axially spaced apart from one another along a longitudinal axis of the catheter body.

11. The method according to claim 10, wherein in method step h), flushing passages are axially produced between two axially adjacent electrodes.

12. The method according to claim 1, wherein the arrangement in method step c) involves helically winding the flexible circuit board around the inner liner.

13. A catheter body produced according to claim 1, wherein the flushing passages have a diameter ranging from 100 µm to 500 µm.

14. The catheter body according to claim 13, wherein a single inner lumen is formed within the inner liner.

15. The catheter body according to claim 13, wherein the flushing passages are distributed radially around a circumference of the catheter body.

16. The method according to claim 2, wherein method step e) is carried out before method step h).

17. The method according to claim 2, wherein method step e) is carried out before method steps f) and g), and method step h) is carried out after method steps f) and g).

18. The method according to claim 3, wherein method step e) is carried out before method steps f) and g), and method step h) is carried out after method steps f) and g).

19. The method according to claim 2, wherein for establishing electrical contact with the electrode in method step g), at least one contact opening is created in the outer insulation in order to selectively establish electrical contact between the electrode and at least one electrical conductor of the flexible circuit board that is accessible via the contact opening.

20. The method according to claim 2, wherein for establishing electrical contact with the electrode in method step g), at least one contact opening is created in the outer insulation in order to selectively establish electrical contact between the electrode and at least one electrical conductor of the flexible circuit board that is accessible via the contact opening.
